(19) United States
(12) Patent Application Publication
(10) Pub. No.: US 2024/0203547 A1
(43) Pub. Date: Jun. 20, 2024

(54) VISUALIZATION OF AND INTERACTION WITH CLINICAL DATA

(71) Applicant: LABORATORY CORPORATION OF AMERICA HOLDINGS, Burlington, NC (US)

(72) Inventors: Enno de Vries, Tower Lakes, IL (US); Jennifer Ennis, Chicago, IL (US); Susan Donahue, Burlington, NC (US)

(21) Appl. No.: 18/542,284

(22) Filed: Dec. 15, 2023

Related U.S. Application Data
(60) Provisional application No. 63/387,862, filed on Dec. 16, 2022.

(51) Int. Cl. G16H 10/60; G06F 3/0483

(52) U.S. Cl. CPC G16H 10/60; G06F 3/0483

(57) ABSTRACT

The present disclosure relates to systems and methods for visualization of data is provided. The systems and methods include receiving a selection of one or more tests, accessing a database containing one or more test results for each of the one or more tests, generating a category tab for each of the one or more tests, and generating data lanes for each of the category tabs and populating each of the data lanes with one or more data points, each of the one or more data points representing the one or more test results received from the database.

### CROSS-REFERENCE TO RELATED APPLICATION

This application claims priority to U.S. Provisional Patent Application No. 63/387,862, filed on Dec. 16, 2022, entitled “VISUALIZATION OF AND INTERACTION WITH CLINICAL DATA” which is hereby incorporated by reference in its entirety.

### FIELD

The present disclosure relates to a method and system for generating a user interface and techniques for the visualization of and interaction with clinical data.

### BACKGROUND

Currently there is a shift from analog records to digital records for ease of access and analysis. The transition of records and requirements for how electronic records can be stored varies by industry. For example, in the medical field, electronic health records (EHR) have been created to maintain patient records. The EHRs are the collection of patient information electronically stored in a digital format. EHRs may include a range of data, including demographics, medical history, medication and allergies, immunization status, laboratory test results, radiology images, vital signs, personal statistics like age and weight, and billing information. However, individual hospitals and hospital partnerships each have their own EHR systems and collection of EHRs.

Frequently, patients have information stored across a variety of EHRs as well as other data service providers. For example, whenever a hospital commissions a test to be performed for a patient, such as a bloodwork panel, the test results are stored in the system utilized by the laboratory. This can include an onsite laboratory sharing usage of the EHR or the laboratory can be a third part laboratory having their own data storage system. With patient data spread across multiple, often disparate systems, it can be difficult for a medical professional to analyze all of the data in convenient and meaningful manner. For example, the patient data related to the medical professional's medical facility can be displayed in one format, test results from laboratories and third-party laboratories may not be compatible with the EHR such that they are provided in paper or other electronic format (e.g., printable document files). Additionally, the data from different sources, or even the same source, cannot be provided in a customizable manner that allows a user to view data in a specified manner or manipulate the data for their particular purposes.

### SUMMARY OF THE INVENTION

Techniques disclosed herein relate generally to medical records systems. More specifically and without limitation, techniques disclosed herein relate to a diagnostic assistant tool that can facilitate decisions of a first entity (e.g., a medical professional) on behalf of a second entity (e.g., a patient) using data from disjunctive sources of data.

In accordance with some embodiments, a method for visualization of data is provided. The methods includes receiving a selection of one or more tests, accessing a database containing one or more test results for each of the one or more tests, generating a category tab for each of the one or more tests, and generating data lanes for each of the category tabs and populating each of the data lanes with one or more data points, each of the one or more data points representing the one or more test results received from the database.

In some embodiments, the receiving the selection of the one or more results includes receiving a selection of a condition preset including the one or more categories. The receiving the selection of the one or more categories can include receiving individually selections of each of the one or more categories. The database can include test results from a plurality of disparate data sources. Each of the one or more data points can be visualized to represent which of the plurality of disparate data sources that data point originates. The method can further include generating a trend line in one of the data lanes, the trend line showing. The method can further include generating a missing observations notification including one or more test results for the one or more categories missing from the database. The data lanes can be oriented vertically to one another and are situated substantially parallel to one another. The method can further include generating, by the processor, a timeline and populating each of the one or more data points within each of the data lanes according to the timeline.

In accordance with some embodiments, a method for updating a graphical user interface (GUI) is provided. The method includes receiving, by a graphical user interface GUI, a selection of one or more tests, generating, by the GUI, visualization of data for each of the selected one or more tests, receiving, by a graphical user interface GUI, an interaction with the visualization of the data for the one or more tests, determining, by a graphical user interface GUI, that additional data is needed, accessing, by a graphical user interface GUI, the additional data from a database, and updating, by the GUI, the visualization of the data based on the interaction and the additional data.

In accordance with some embodiments, a system is provided. The system includes one or more processors and a memory coupled to the one or more processors, the memory storing a plurality of instructions executable by the one or more processors, the plurality of instructions comprising instructions that when executed by the one or more processors cause the one or more processors to perform the method.

In accordance with some embodiments, a non-transitory computer-readable memory storing a plurality of instructions executable by one or more processors, the plurality of instructions includes instructions that when executed by the one or more processors cause the one or more processors to perform the method.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

FIG. 1A is a block diagram of a credential enabled reference for a visualization system in accordance with various embodiments.

FIG. 1B is block diagram illustrating some of the functional components for a visualization system in accordance with various embodiments.

FIGS. 2A, 2B, and 2C are example graphical user interfaces for performing a selection in accordance with various embodiments.

FIGS. 3A, 3B, 3C, 3D, 3E, 3F, 3G, 3H, 3I, and 3J are examples of graphical user interfaces for visualizing data in accordance with various embodiments.

FIG. 4 is a flow diagram for generating a graphical user interface for visualizing data in accordance with various embodiments.

FIG. 5 is an example of a graphical user interface for creating a custom condition preset in accordance with various embodiments.

FIG. 6 is a flow diagram for interacting with visualized data within a graphical user interface in accordance with various embodiments.

FIG. 7 block diagram of a computing device for use in a visualization system in accordance with various embodiments.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

The present disclosure relates to a method and system for the organization, transformation, and visualization of aggregated quantitative data. The present disclosure is designed to collect and/or access data for a combination or local and disparate data sources, organize the data in a meaningful manner, and present a visualization of the data in a way that is advantageous for a user. The visualization of the data can be dictated on a combination of factors, including, but not limited to, user preferences, types of data, sources of the data, etc. The visualization can be provided to a user in a way that lets the user draw conclusions by presenting one or more sets of related data in a visually comparable manner.

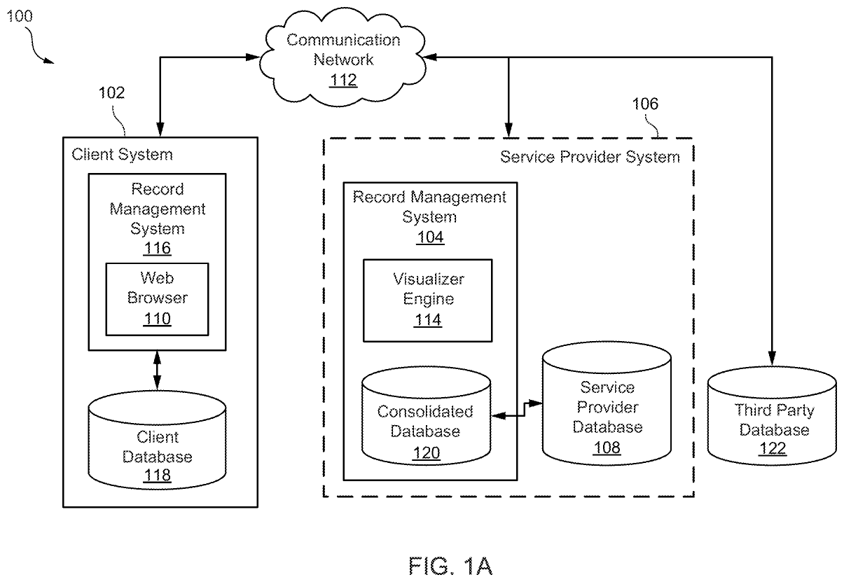

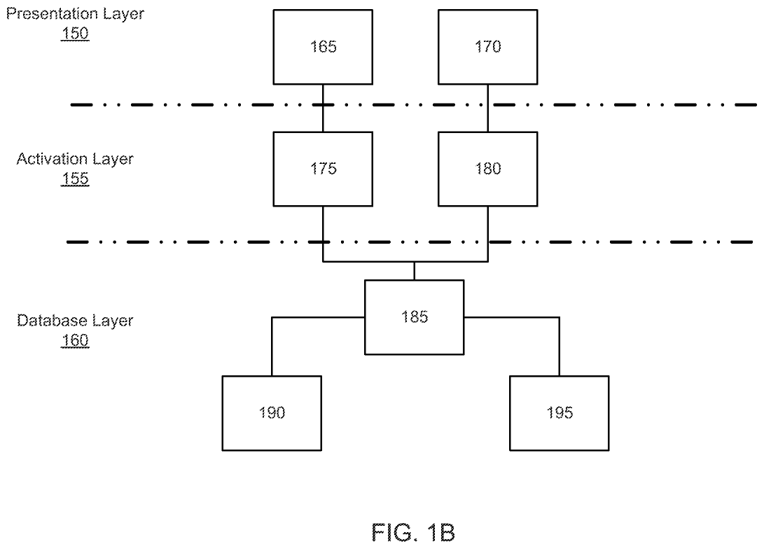

FIG. 1A depicts an illustrative system 100 for implementing steps in accordance with aspects of the present disclosure. In particular, FIG. 1A depicts a system 100 that can include a client system 102, a record management system 104, and a service provider system 106. The record management system 104 can be part of the service provider system 106 or a separate system and/or service provider. Each of the client system 102, the record management system 104, and the service provider system 106 can include a combination of hardware and software configured to carry out aspects of the present disclosure. In particular, the client system 102, the record management system 104, and the service provider system 106 can include a computing system with specialized software and databases designed to provide graphical user interfaces designed to present interactive visualizations of data to a user. As would be appreciated by one skilled in the art, each of the client system 102, the record management system 104, and the service provider system 106 can include a single computing device, a collection of computing devices in a network computing system, a cloud computing infrastructure, or a combination thereof. For example, the computing devices can include any combination of servers, personal computers, laptops, tablets, smartphones, etc. In some embodiments, different components or elements of the client system 102, the record management system 104, and the service provider system 106 can include their own computing devices.

In some embodiments, the client system 102 can include a computing device having software installed thereon for accessing data provided by other components within the system 100. For example, the client system 102 can include a computing device having a web browser 110 configured to access web pages and download content on a network such as the communication network 112 (e.g., Internet). In some embodiments, the client system 102 can include or otherwise can be communicatively coupled to a record management system 116 and a client database 118. The record management system 116 and the client database 118 can be designed to manage and store records for access by the client system 102. The records can include any combination of records, including for example, electronic medical records (EMR) and/or electronic health records (EHR) and software to manage and access the EMR/EHR. In such an example, the client system 102 can be a system operated by a medical service provider and the records stored within the client database 118 can be patient records for that medical service provider. Similarly, the client system 102 can also be configured to be communicatively coupled to the record management system 104 and/or the service provider system 106 and can include software for accessing, reading, writing, etc. data on the record management system 104 and/or the service provider system 106. The client system 102 can either access the record management system 104 through the communication network 112, it can be directly coupled/networked to the record management system 104, service provider system 106, or both.

Similar to the client system 102, the service provider system 106 can include any combination of systems that host third party applications or services to be utilized by the client system 102, for example, as discussed in greater detail with respect to U.S. 63/387,859, which is incorporated by reference herein in its entirety. In some embodiments, the service provider system 106 can be a system of a clinical laboratory where tests are carried out on clinical specimens to obtain information about the health of a patient to aid in diagnosis, treatment, and prevention of disease. For example, the tests can include medical tests to evaluate a person's physical or mental health such as tests on blood or urine samples, tests on tissue specimens, tests on other biological materials, psychological or physiological evaluation, physical fitness, etc. The tests can be conducted using any combination of systems or methods. For example, the tests can include physical measurement, electromechanical analysis, analytical chemistry, molecular diagnostics, user evaluation, microbiology, serology or immunology, medical imaging, and the like. For purposes of this disclosure, the term tests will be used synonymously with any type of clinical laboratory test.

In some embodiments, the service provider system 106 can be a laboratory that includes an accessioning and processing system for receiving, sorting, and processing the one or more specimen/samples for testing purposes. All of the received, stored, and derived information related to accession data, patient data, provider data, etc. can be stored in the service provider database 108. In some embodiments, each diagnostic testing order, including one or more tests on a specimen/sample, is linked to an accession identifier. Similar to the client database 118, the service provider database 108 can include any combination of computing devices configured to store and organize a collection of data. In some embodiments, the service provider system 106 can aggregate and store test data from a plurality of disparate data sources to be conveyed to a client system 102.

Once all or a portion of the testing associated with an accession identifier is completed, in some embodiments, the service provider system 106 can provide the results back to the requesting entity. For example, if a user on the client system 102 submitted the diagnostic testing order to the laboratory (e.g., via service provider system 106), then the results can be provided to the user. The results can be provided to the requesting user using any combination of media. For example, lab results can be provided via a web application interface, email, through the web browser 110, web application, a short message service (SMS), an app push notification, snail mail, fax, etc. The results can include any combination of test results, test panel results, test chemistry results, analytic results, diagnostic procedures results, etc. Additionally, the results can include various levels of details depending on the type of tests being performed and information being conveyed. For example, results can include laboratory results, physiological results, mental results, physical results, etc. Additionally, the test results can be conveyed using any combination of informational presentations. For example, the test results can be any combination of measured values, expected values, graphs, color coding, detailed descriptions, etc. For purposes of this disclosure, the results will be used synonymously with any of these types of test results.

Continuing with FIG. 1A, the service provider system 106 can include or otherwise be communicatively coupled to the record management system 104. If separate from the service provider system 106, the record management system 104 and the consolidated database 120 can share and/or receive some data with third parties, such as the service provider system 106. In some embodiments, the record management system 104 and the consolidated database 120 can aggregate and deduplicate data from a collection of databases, for example, any combination of the client database 118, the service provider database 108, and any third-party sources (e.g., third party database(s) 122). The data aggregation and deduplication can be performed using any combination of techniques, for example, as discussed in greater detail with respect to U.S. 63/387,859, which is incorporated by reference herein in its entirety. The record management system 104 can interact with the consolidated database 120 utilizing a given database model configured to interact with a user for analyzing the stored and consolidated data. The consolidated database 120 can include any combination of computing devices configured to store and organize a collection of data. For example, consolidated database 120 can be a local storage device on the service provider system 106, a remote database facility, or a cloud computing storage environment.

In some embodiments, the combination of the record management system 104 and the consolidated database 120 includes electronic medical records (EMR) and/or electronic health records (EHR) and software to manage and access the EMR/EHR. When storing, accessing, or sharing, sensitive or protected data, the record management system 104 and the consolidated database 120 can be configured to protect the information. For example, the record management system 104 and the consolidated database 120 can be configured to de-identify the data, encrypt the data, or a combination thereof. The consolidated database 120 may be a proprietary or specialized system that has targeted functionality depending on the needs of the user/industry. For example, traditional EMR/EHR systems are frequently limited on their ability to aggregate information from multiple data sources (e.g., the client database 118, the service provider database 108, third party database 122, etc.) and visualize the data in a meaningful way.

In some embodiments, the record management system 104 can include a visualization engine 114. The visualization engine 114 can be configured to collect, aggregate, or otherwise access information from the consolidated database 120 and/or from a plurality of data sources. In some examples, the visualization engine 114 can look for and address non-conformities in information. For example, the diagnostic tool can confirm that aspects, such as units, of the information comply with industry standards for the data. If a portion of the information does not conform with the rest of the information, the diagnostic tool can modify the portion of the information so that the portion is more consistent with the rest of the information. For example, if the portion of the information includes units that do not match industry standards for units, the diagnostic tool can perform a unit conversion on the portion of the information. For example, the visualization engine 114 can access data from the client database 118, the third-party database(s) 122, and/or the service provider database 108. The data from different data sources can be combined in a manner for use by the visualization engine 114, for example, as discussed in greater detail with respect to U.S. 63/387,859, which is incorporated by reference herein in its entirety. In some embodiments, the visualization engine 114 can organize the data from one or more data sources to be organized and combined into a desirable visualization for user observation and interaction. For example, the visualization engine 114 can organize data from all data sources into predetermined categories, such as disease type, conditions, symptoms, or customized reports, charts, graphs, etc. in a manner that is easy for a user to understand and draw conclusions from. In some examples, the visualization engine 114 can present the data in a unified form within customized reports, charts, graphs, etc. For example, even when the data is collected from multiple sources and has mismatched units, the visualization engine 114 can automatically perform unit conversions on portions of the data so that all relevant data may be presented within customized reports, charts, graphs, etc. with unified units for simple comparison of the data.

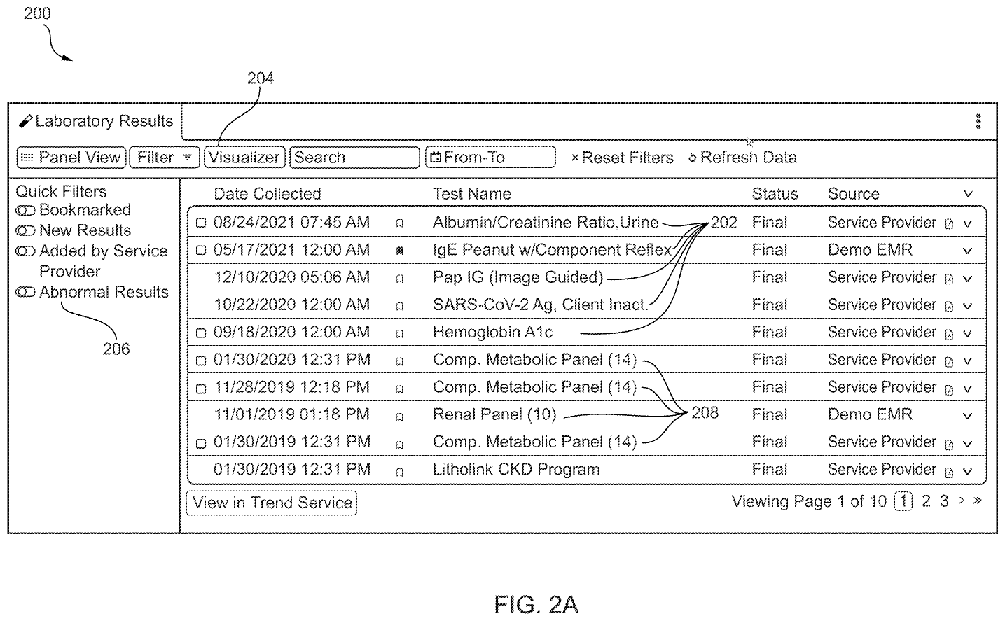

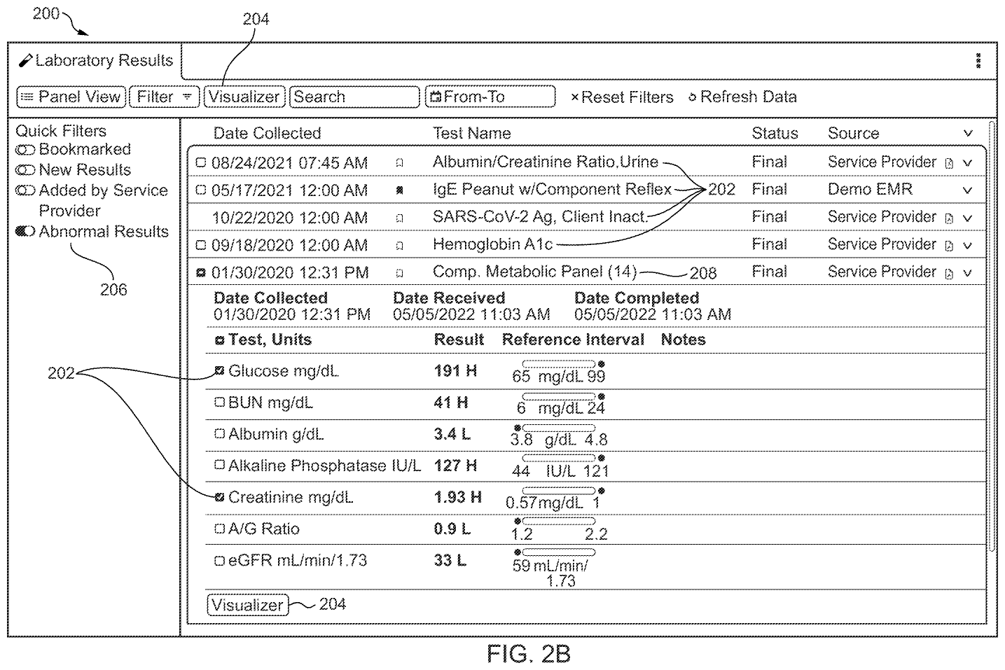

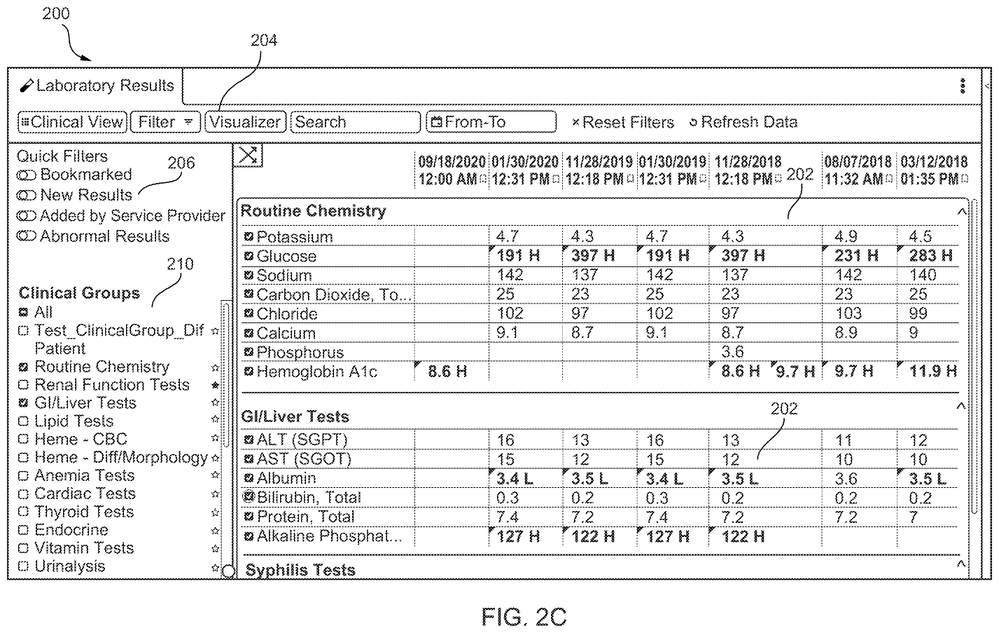

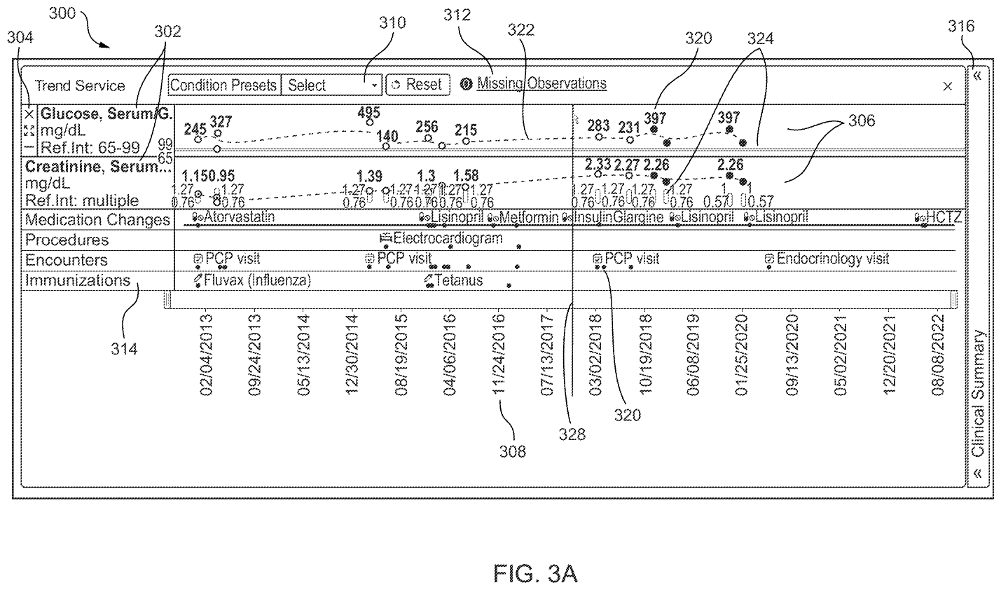

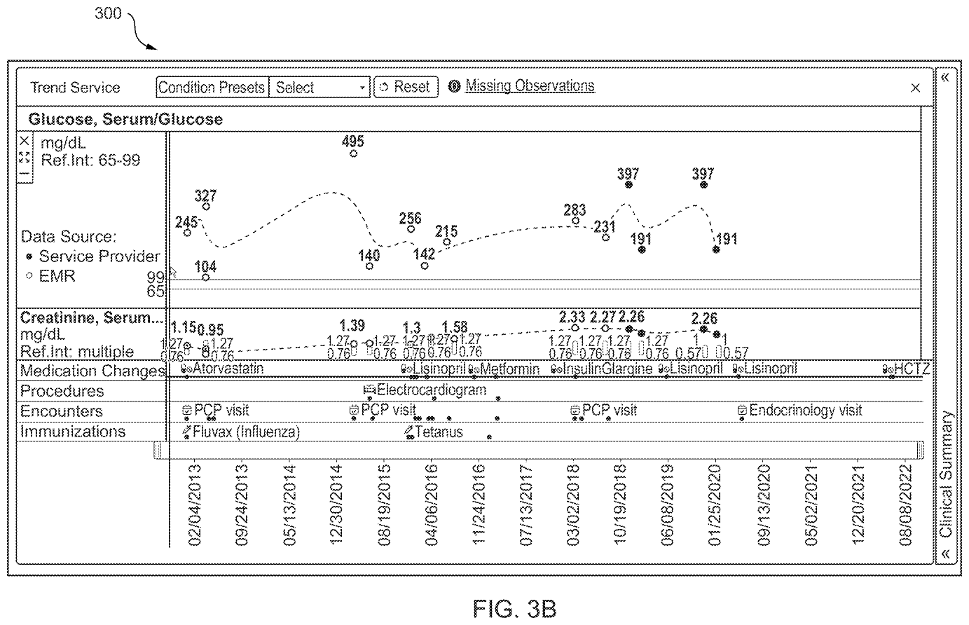

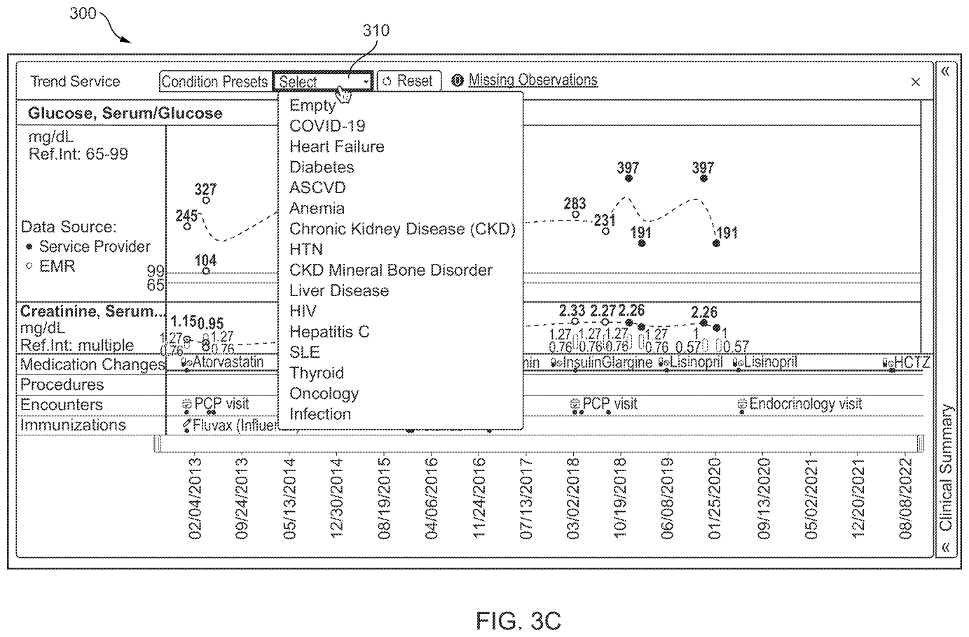

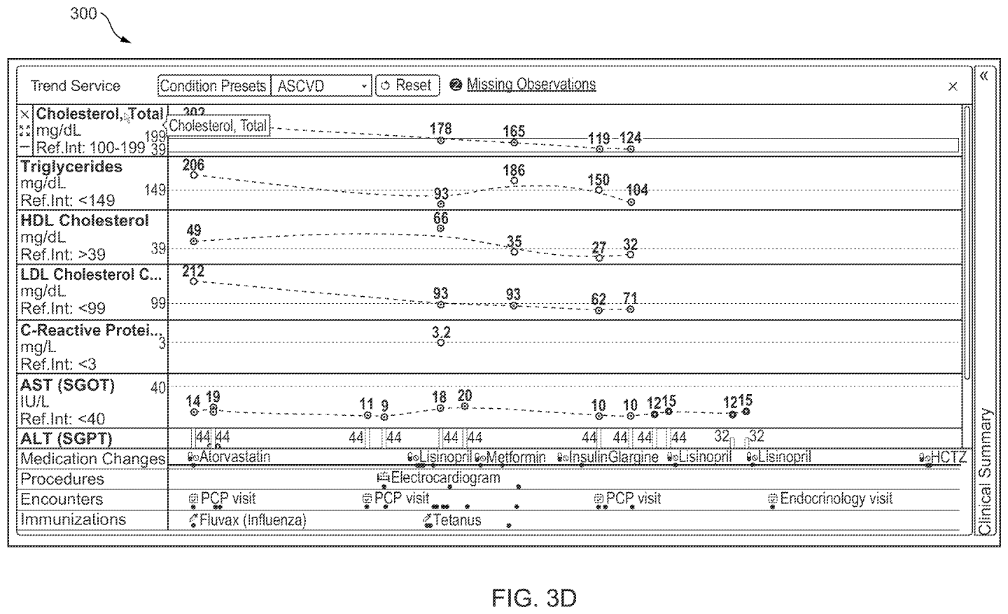

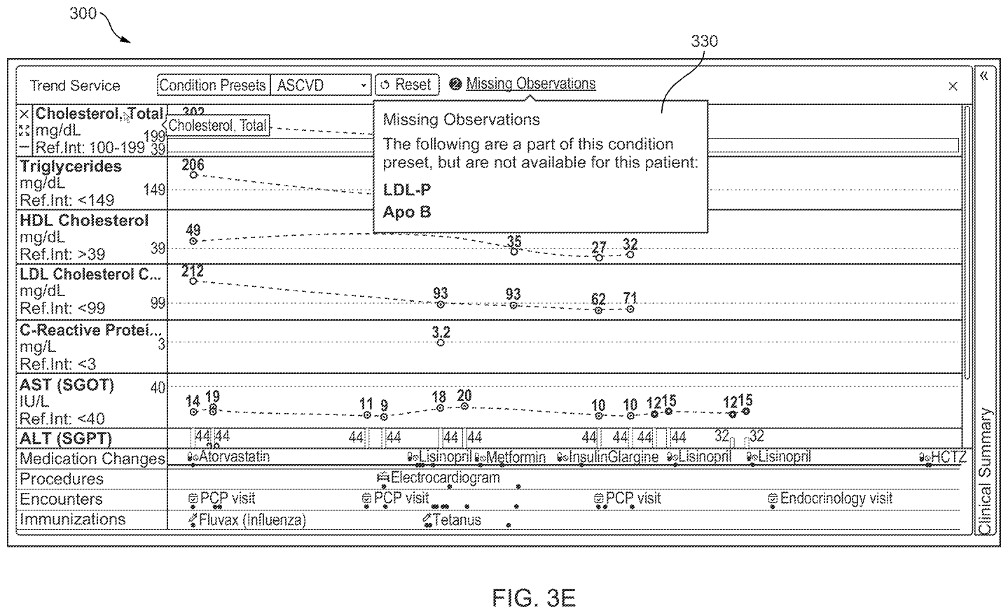

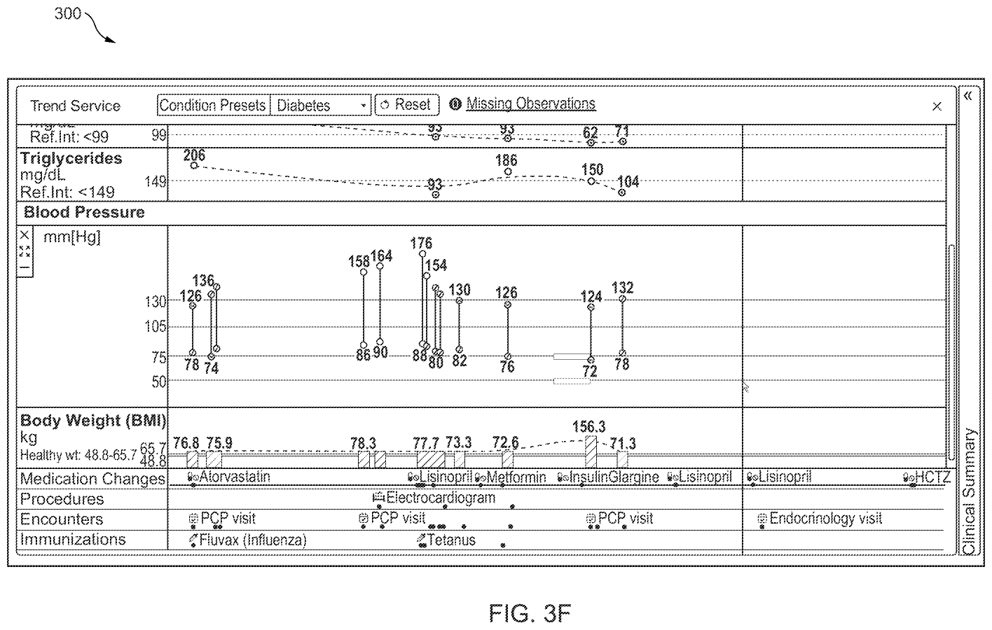

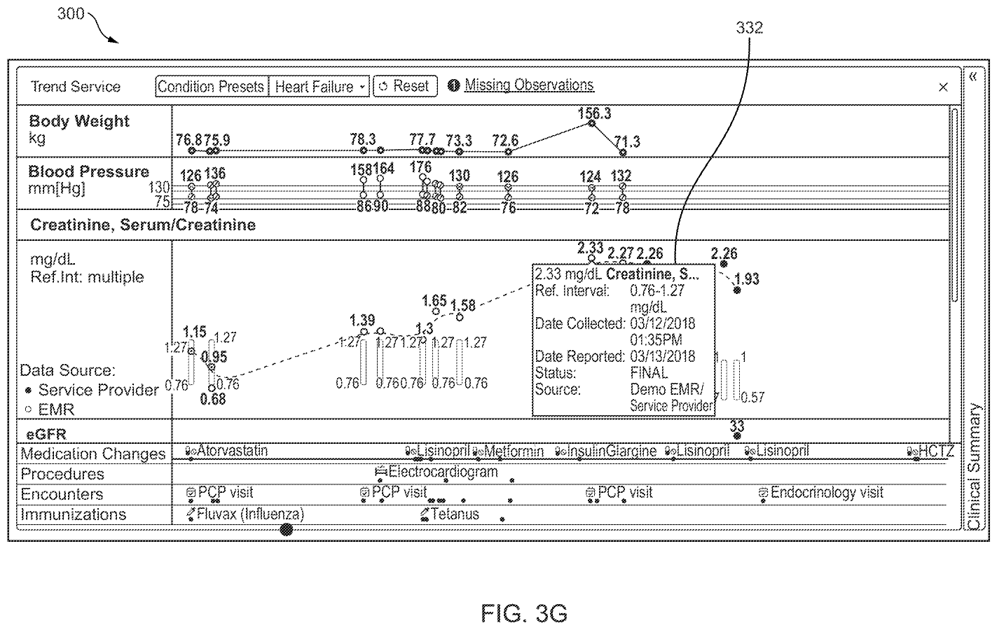

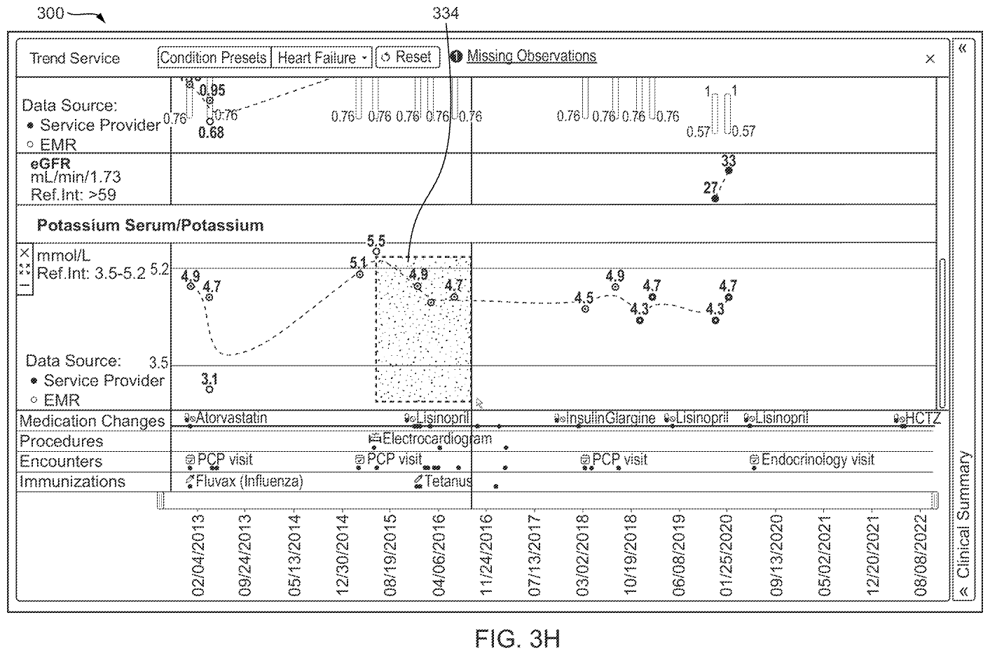

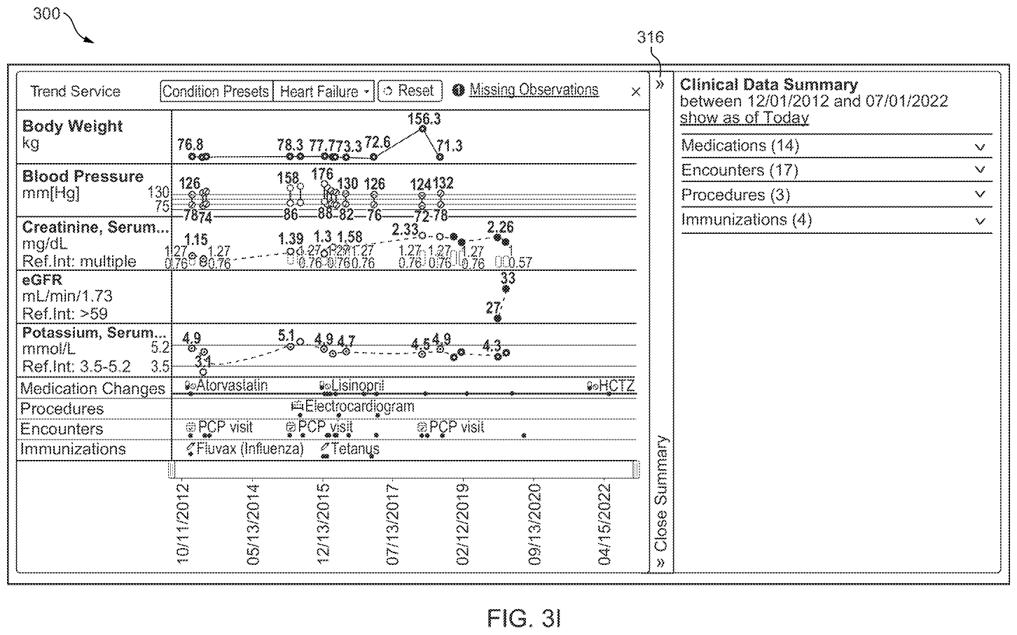

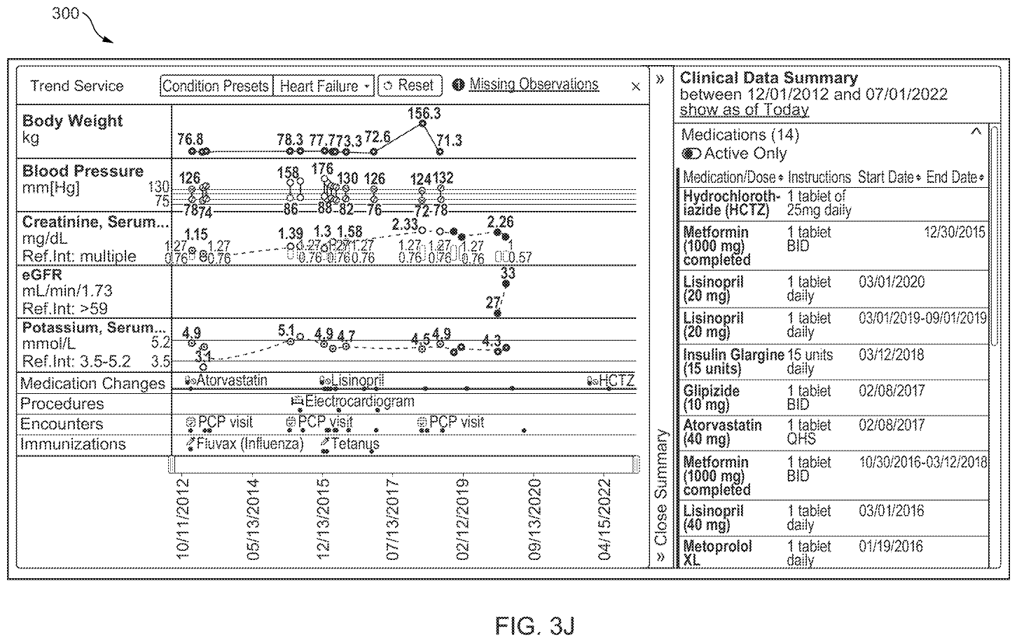

Referring to FIG. 1B, a block diagram illustration of some of the functional components for providing the visualization of data, for example, by the visualization engine 114, is provided. In some embodiments, the visualization architecture can include three layers: a presentation layer 150, an application layer 155, and a database layer 160. The presentation layer 150 can include a plurality of user interfaces 165, 170 through which users (e.g., practitioners or administrators) review medical information for a patient(s). The user interfaces 165, 170 can include any combination of user interfaces in accordance with the present disclosure, including but not limited to GUIs 200, 300 as described with respect to FIGS. 2A-3J. The user interfaces 165, 170 can reside on one or more workstations or on one or more personal computers. In general, user interfaces 165, 170 can reside on any computational system, and although two GUIs are shown in FI. 113 it should be understood that any number of GUIs can be developed and provided in accordance with the aspects described herein.

In some embodiments, the user interfaces 165, 170 can be coupled to one or more application servers within the application layer 155. Application servers 175 and 180 can implement operations to facilitate visualization of data aggregated from a plurality of data sources (e.g., within the consolidated database 120). In doing so, application servers 175 and 180 can communicate and process information between user interfaces 165, 170 and the communication network. In various embodiments, the application servers 175 and 180 facilitate visualization of data aggregated from a plurality of data sources through a set of mechanisms described herein. Application servers 175 and 180 may be located at a number of locations in a distributed computing system, including at a computational server or a database server, and may be in communication with any of the user interfaces in the presentation layer 150.

In some embodiments, the application servers 175 and 180 can be coupled to a database management system 185 (e.g., visualization engine 114 and/or record management system 104 as discussed with respect to FIG. 1A) within the database layer 160. Database management system 185 can be any type of custom-made or commercially available database system for managing storage and retrieval of data. In some embodiments, database management system 185 is configured to collect, aggregate, and format data from a plurality of disparate database servers 190, 195 (e.g., client database 118, service provider database 108, third party database(s) 122), and the like. Exemplary database servers include without limitation those commercially available from Oracle, Microsoft, Sybase, IBM and the like. Database management system 185 can include or otherwise be communicatively coupled with databases 190, 195 (e.g., record management system 104 and record management system 116 as described with respect to FIG. 1A), The data received from each of the databases 190, 195 can be stored and formatted in a common format such that common data can be aggregated together and used by the visualization engine 114. For example, all data for Jane Doe can be aggregated into a data structure, deduplicated, and tagged with a data source.

Returning to FIG. 1A, in some embodiments, the visualization engine 114 can be configured to generate customizable graphical representations of quantitative data to replace conventional individually created disparate reports. For example, the visualization engine 114 can generate a graphical user interface (GUI) containing one or more displays showing an individual data set, an aggregation of historical data sets, a collection of aggregated historical data sets, etc. The data sets can include any combination of data. For example, the data sets can be related to medical data, test data, etc. which can include test results, biometric data, etc. The data sets can also include supplemental data that correspond to the data sets. For example, the supplemental data can include ideal data ranges, target values, high or low thresholds, contextual information, etc. The visualization engine 114 can be configured to review, interpret, analyze, modify, and/or determine how to best visualize the data.

Each component within the system 100 can include a plurality of devices configured to communicate with one another over a communication network(s) 112. In some embodiments, the client system 102, the record management system 104, and the service provider system 106 can establish a connection and communicate over communication network(s) 112 to carry out aspects of the present disclosure. As would be appreciated by one skilled in the art, the communication network(s) 112 can include any combination of known networks. For example, the communication network(s) 112 may be a combination of a mobile network, WAN, LAN, or other type of network. The communication network(s) 112 can be used to exchange data between the client system 102, the record management system 104, the service provider system 106, and/or to collect data from additional sources.

Continuing with FIG. 1A, a combination of hardware and software that make up the system 100 can be specifically configured to provide a technical solution to a particular problem utilizing an unconventional combination of steps/operations to carry out aspects of the present disclosure. In particular, the system 100 can be designed to execute a unique combination of steps to provide a novel approach to aggregating, organizing, formatting, and visualizing information to a user in an advantageous manner. In conventional systems, an abundance of information may be available, however, conventional systems may lack a mechanism for conveying information to a user in a combined manner. Typically, information can be viewed independently from other forms of information in an original format. For example, a diagnostic test result for a patient can be viewed as an independent report but the diagnostic test result may not typically be viewable in relation to previous test results of a same type much less in relation to previous test results of other but potentially related types of tests. Often, an agent for accessing the information can be limited in an ability to convey the information in any format other than an originally received format. For example, EMRs or EHRs for a record management system 104 may only be able to display information in a format received from internal or external laboratories provided reports. The system 100 of the present disclosure can provide an improved system and methodology for combining, organizing, and visualizing data to a user in a meaningful way.

Referring to FIGS. 2A-2C, example graphical user interfaces 200 for displaying test information, diagnostic information, clinical data, etc. are depicted. The GUI 200 can include any combination of user interface elements such as tabs, menus, selectors, search bars, etc. to enable interaction with a user for implementing the present disclosure. For example, as depicted in FIG. 2A, the GUI 200 can include various filters, a search bar, date range selection, refresh/reset buttons, one or more visualizer buttons 204, and quick filters 206. General usage of the various components in GUI 200 can operate in a manner consistent within the art. In some embodiments, the GUI 200 can provide a list of tests and/or test results. The list of tests and/or test results can include an ordered list of tests 202 performed for one or more subjects (e.g., patients). The tests 202 can be ordered based on any combination of criteria, for example, by date performed, alphabetically, viewed/unviewed, date collected, etc.

In some embodiments, the data within the GUI 200 can be specific to a particular patient record, collection of patient records, system records, etc. For example, all the tests 202 performed can be specific to a single subject such as patient Jane Doe. As shown in FIG. 2A, the list of tests 202, within the GUI 200, can include basic information including date collected, test name (e.g., test performed), status, source, etc. In some embodiments, entries within the list of tests 202 can be received from a plurality of sources to be accessed and viewed by a user. For example, each of the entries from the list of tests 202 can be populated from data sources such as a consolidated database 120, a client database 118, third-party service provider databases (e.g., third party database(s) 122), public repositories, or a combination thereof. The tests 202 illustrated in FIG. 2A are provided as an example but it should be understood the tests 202 can include any combination of clinical laboratory tests and are not limited to any specific tests shown in the figures.

Referring to FIGS. 2B and 2C, in some embodiments, users can interact with the GUI 200 to review additional data available for various tests within the list of tests 202. In some instances, the list can also include test panels 208 including a group of tests 202 (as shown in FIG. 2B for a comprehensive metabolic panel). A test panel can be a predetermined group of tests (e.g., a group of genetic markers, analytes, biomarkers, etc.). However, the test panels 208 provided in GUI 200 may not be disease specific. For example, the comprehensive metabolic panel includes tests that are relevant to hundreds of different diseases. To facilitate a clearer understanding of test results, data provided in GUI 200 can be filtered to better identify information that may be useful to the user. In some embodiments, the GUI 200 can include one or more predetermined quick filters 206. The quick filters 206 can be selected to quickly filter the tests 202 displayed within the GUI 200 according to selected criteria. For example, as shown in FIG. 2B, the quick filters 206 can include toggles that limit a display of the tests 202 including filters for bookmarked tests, new test results, tests added by a third-party provider, abnormal test results, etc.

Referring to FIG. 2B, the GUI 200 is shown after some interaction with a user. As shown, a user has selected a quick filter 206 for abnormal results, limiting the tests 202 and/or test panels 208 displayed to be those that have been determined to have an abnormal result or to include at least one test within a test panel determine to be abnormal (e.g., an abnormal analyte level). Additionally, a user selection of a test panel 208 can cause the GUI 200 to open a drop-down box or expanded window from that selection, which shows all or a portion of various tests 202 analyzed or measured as part of a test panel 208 (e.g., in this instance, the comprehensive metabolic panel). For example, as shown in FIG. 2B, a selection of the comprehensive metabolic panel shows seven of fourteen specific tests that were determined as having an abnormal result, including abnormal results for Glucose, BUN, Albumin, Alkaline Phosphatases, Creatinine, A/G Ratio, and eGFR. The tests 202 shown in the drop-down box or expanded window can include any combination of information. For example, as depicted in FIG. 2B, the tests 202 can include columns for test type, measurement units, test result, reference intervals, graphical flags or notifications, notes, etc. In some embodiments, the tests 202 and test panels 208 can be selectable by receiving an input from a user. For example, the GUI 200 can receive a signal from a user selecting one or more check boxes associated with one or more of the tests 202 and test panels 208. The tests 202 and test panels 208 illustrated in FIG. 2B are provided as an example but it should be understood the tests 202 and test panels 208 can include any combination of clinical laboratory tests and are not limited to any specific tests shown in the figures.

Referring to FIG. 2C, another example of the GUI 200 is shown after some interaction with a user. In some embodiments, the GUI 200 can include one or more clinical groups 210. Each of the clinical groups 210 can include a list of tests, predetermined groups of tests, test panels, a combination thereof, etc. Similar to tests 200 and test panels 208, a user can select each of the clinical groups 210. As shown in FIG. 2C, a user has selected two clinical groups 210 from a list of predetermined clinical groups 210. Similar to FIG. 2B, selection of the two clinical groups 210 can trigger the GUI 200 to load two predetermined lists of tests 202 and/or test panels 208 to be displayed for selection or deselection by a user. For example, as shown in FIG. 2C, the GUI 200 receives a user selection for Routine Chemistry and GI/Liver Tests clinical groups 210, which causes the subsets of tests 202 for the Routine Chemistry and GI/Liver Tests to be displayed. The subsets of tests 202 can be selected by default and the user can deselect if they would like to customize the tests to be visualized, or vice vera. For example, the GUI 200 can receive a signal from a user selecting or deselecting one or more check boxes associated with one or more of the clinical groups 210 and tests 202 (or test panels 208).

Referring to FIGS. 2B and 2C, in some embodiments, the user can select specific tests 202 and/or test panels 208 to be visualized along with other specified tests 202 and/or test panels 208. The tests 202 and/or test panels 208 being visualized together may not be compatible or similar types of data. For example, the compared tests 202 and/or test panels 208 can be non-comparable value sets, different ranges, different ratios, etc. Unlike as provided by the present disclosure, conventional data series may be plotted on top of one another, regardless of compatibility, and may not be organized in a useful manner for a viewing user to draw any conclusions.

In some embodiments, the selection of one or more of the tests 202 and/or test panels 208 and selection of a visualizer button 204 can launch a graphical user interface 300 (see, e.g., FIG. 3A) for visualizing the selected test data included within a collection of data sources. For example, if the Glucose and Creatinine tests are selected and the visualizer button 204 is selected, then the GUI 300 will be launched. The GUI 300 can show a visualization of all test data associated with a patient related to Glucose and Creatinine measurements. A similar functionality can be provided for entire test panels 208 and is not intended to be limited to a selection of a subset of tests 202. For example, selection of the Comprehensive Metabolic Panel and the visualizer button 204 can cause the GUI 300 to launch, showing the visualization of all data associated within the Comprehensive Metabolic Panel test results.

Referring to FIG. 3A, in some embodiments, a system (e.g., system 100) can be configured to provide a graphical user interface 300 presenting an interactive visualization of a graphical representation of one or more selected data sets. The data sets can include any combination of data, for example, the tests 202, test panels 208, the clinical groups 210, or any combination thereof as provided in GUI 200. The GUI 300 can include any combination of user interface elements such as tabs, menus, selectors, search bars, reset button, etc. to enable interaction with a user to implement the present disclosure. The GUI 300 can include different basic graphical user interface controls to provide functionality consistent in the art. For example, user selection of a reset button can trigger the GUI 300 to reset to a default view, removing any changes the user may have made to the GUI 300 from an initial/preset state. In some embodiments, as depicted in FIG. 3A, the GUI 300 can include tabs 302 (corresponding to selected tests 202, test panels 208, and/or the clinical groups 210), a tab modifier 304, one or more data lanes 306, a timeline 308, a condition presets drop down menu 310, a missing observations notification 312, patient medical history tabs 314, a summary tab 316, etc. In some embodiments, the system 100 can generate the GUI 300 based on data selections from GUI 200.

In some embodiments, a tab 302 can be generated and populated for each selected test 202, including tests within selected test panel 208 and/or the clinical group 210, provided from GUI 200. Continuing the example provided in FIGS. 2A-2C, a selection of Glucose and Creatinine tests in GUI 200 can trigger the GUI 300 to create two tabs 302, one for each of the Glucose and Creatinine tests, as shown in FIG. 3A. In some embodiments, the tabs 302 can be positioned substantially vertically to one another, creating a stack of tabs, for example, as depicted in FIG. 3A. The selected tabs 302, can include basic information about the tests displayed in the GUI 300. For example, the tabs 302 can include a title of a displayed test, the measurements for the displayed test, ideal ranges for the displayed test, etc. The tabs 302 can also include other types of data and may not be limited to tests. For example, the tabs 302 can include tabs for body weight (BMI), blood pressure, heart rate, etc.

In some embodiments, the tabs 302 can include the tab modifier 304. The tab modifier 304 can be configured to modify the tabs 302 and related content (e.g., data lanes 306). For example, the tab modifier 304 can include options to close, hide, or delete the tabs 302 and related content within the GUI 300, move, or reorder the tabs 302 and related content within the GUI 300, and restore, expand, and minimize the tabs 302 and related content within the GUI 300. In some embodiments, if a user has made changes using the tab modifier 304 (e.g., rearranging, resizing, hiding, etc. the various tabs 302) and wants to restore the original view of the tabs 302, the reset button can be selected to trigger the GUI 300 to load the default view.

In some embodiments, similar to the tabs 302, the data lanes 306 can be generated and populated for each selected test 202, including a selected test from within test panel 208 and/or clinical group 210, provided from GUI 200. Continuing the example above, the selection of Glucose and Creatinine tests in GUI 200 can trigger the GUI 300 to create two data lanes 306, one for each of the Glucose and Creatinine tests, as shown in FIG. 3A. Each of the data lanes 306 can be associated with each of the tabs 302 and can extend in a horizontal direction from each of the tabs 302.

In some embodiments, the data lanes 306 can include a graphical visualization of data related to test results. For example, the data lanes 306 can include graphs or plots corresponding to all test results for the selected test. In some embodiments, the data lanes 306 can include a plurality of data points 320 associated with a particular patient, EMR/EHR, entity, or group. For example, the data lanes 306 can include all the test data for a patient that have been generated within a medical history of the patient. For example, as shown in FIG. 3A, the data lane 306 associated with a Glucose tab can include all Glucose test results for that patient (e.g., Jane Doe) over a period of time (e.g., between Feb. 13, 2004 and Jan. 25, 2020).

In some embodiments, visualization of data within the data lanes 306 can include data points 320, a trend line 322, ideal ranges 324, or any combination thereof. Each data lane 306 can include data points 320 that represent a test result provided at some point in time. For example, as shown in FIG. 3A, the data lane 306 for the Glucose tests includes fourteen data points for fourteen separate tests that were conducted between Feb. 13, 2004 and Jan. 25, 2020. The data points 320 can be represented by any combination of graphical visualizations. For example, the data points 320 can be represented by circles with data values associated therewith, as shown in FIG. 3A. Additionally, the data points 320 can include a graphical representation of the data source. For example, as shown in FIG. 3A, the data points 320 can include a hollow outline circle for a first data source (e.g., client database 118) and a solid-colored circle for a second data source (e.g., service provider database 108). The data points 320 can also be color coded to reflect certain characteristics. For example, the data points can be color coded to indicate a normal or within an ideal range result (e.g., green), or abnormal or outside of an ideal range results (e.g., red).

In some embodiments, a trend line 322 can be generated within a data lane 306 to show a continuous curve that represents a general trend of the data points 320. The trend line 322 can be generated using any combination of techniques. For example, the trend line 322 can be generated using a smoother function as part of a polynomial equation with a set tension to allow outliers to be less influential. The trend line 322 can graphically provide additional context to a user in making one or more decisions or conclusions. The trend line 322 may not necessarily connect to each of the data points 320 but can instead flow and adjust based on each of the data points 320 over time (e.g., timelines 308). For example, the trend line 322 can adjust for outliers to show a relative distribution since some test results are daily values or outliers (e.g., patient just ate sweet cake so a higher glucose value was measured). A use of the trend line 322 can be data type dependent, such that tests or other measurements (e.g., biometrics, vitals, etc.) may not have a trend line 322 because the tests or other measurements may have fixed values. For example, a data lane 306 for body weight would not require a trend line 322 because body weights can be fixed values and are not necessarily impacted by other factors, such as a time that the body weight is measured.

In some embodiments, ideal ranges 324 can be plotted proximate to the data points 320. The ideal ranges 324 can include target ranges for a typical individual or the target ranges for the target individual associated with the data points 320. The ideal ranges 324 can be fixed values or the ideal ranges 324 can be variable and can adjust according to a number of factors. For example, the ideal ranges 324 can vary depending on measurement tools being implemented, an individual or specimen being tested, etc. When the ideal range 324 values change within a data lane 306, relative data to the reference range can also change accordingly. As shown in FIG. 3A, the ideal range 324 for a data lane 306 associated with the glucose tab 302 can begin at a range of 0.76-1.27, then the ideal range can changes after a last two results to be 0.57-1. Fixed range values can be represented differently than variable range values. For example, as shown in FIG. 3B, a range of 65-99 for the data lane 306 associated with the glucose tab 302 can be visualized as a consistent horizontal bar, whereas variable ranges for a data lane 306 associated with Creatine can be visualized as separate vertical bars rendered with each respective data point.

In some embodiments, if no data is available for a selected tab 302 or data lane 306, then no data will be generated and a corresponding tab 302 and data lane 306 will not be visualized. Instead, as discussed in greater detail below, any missing data associated with a selected test can be identified under the missing observations notification 312. A user can select or view the missing observations 312 notification to help make one or more decisions with respect to a particular patient. For example, the user can recommend that the patient undergo a blood pressure test to remedy the missing information when a blood pressure test is one of the selected tests identified under the missing observations notification 312.

Referring to FIG. 3B, in some embodiments, the one or more data lanes 306 can be expanded to provide an enhanced view of the plotted data points 320 for the data lane 306 as well as additional details about the data. The data lanes 306 can be expanded in any combination of methods, for example, in response to receiving a selection of a tab modifier 304 associated with that data lane 306. User interaction with an expanded data lane 306 can be similar to that of a non-expanded data lane 306, however, the expanded data lane 306 may include additional details or functionality.

Returning to FIG. 3A, in some embodiments, each of the data lanes 306 can be plotted against the timeline 308. The timeline 308 can be provided with constant values associated with each of the data lanes 306, such that the data points 320 populated within the data lanes 306 can be adjusted to be consistent with the timeline 308. In some embodiments, the timeline 308 can include a time indicator 328 reflecting a select time or time period throughout each of the data lanes 306. For example, as depicted in FIG. 3A, the time indicator 328 can be a vertical line temporally aligned and extending perpendicularly from the timeline 308 and extending through each of the one or more data lanes 306. The time indicator 328 can move based using any combination of mechanisms, for example, the time indicator 328 can follow a mouse cursor, can move in response to a click and drag routine, move in response to a double click, etc.

Referring to FIG. 3C, in some embodiments, each of the tabs 302 and the corresponding data lanes 306 can be populated based on a selection of a condition. The condition can be, for example, selected from a condition presets drop down menu 310. In some examples, the selection can occur in a different computing environment and the condition presets drop down menu 310 may not be included in GUI 300. For example, the selection can occur in a module associated with assisting with clinical decisions and the GUI 300 can be redirected to the module to receive the selection of the condition. Upon receiving a selection of one of the condition presets, the GUI 300 can compare characteristics for the selected condition preset and compare the characteristics against one or more files (e.g., patient files, EMR/HER, etc.) to see what results are included or missing but should be included. The included results can be visualized within the GUI 300 (e.g., loading tabs 302 and populating associated data lanes 306 with the included data points 320) and any missing data for categories associated with the selected condition preset can be included within the missing observations notification 312. The condition presets drop down menu 310 can include a plurality of predefined conditions that include one or more characteristics including test values or other measured values associated therewith. For example, as shown in FIG. 3C, the GUI 300 can include condition presets for COVID-19, Heart Failure, Diabetes, ASCVD, Anemia, Chronic Kidney Disease (CKD), HTN, CKD Mineral Bone Disorder, Liver Disease, HIV, Hepatitis C, SLE, Thyroid, Oncology, Infection, etc., each having an associated predetermined set of tabs 302. The condition presets drop down menu 310 provided in FIG. 3C is for illustrative purposes only and is not intended to be limiting, such that any combination of diseases or conditions can be included within the condition presets drop down menu 310. The conditions within the condition presets drop down menu 310 can be based on predetermined guidelines and/or clinical advice-based profiles for monitoring and reviewing certain collections of test result sets.

In some embodiments, the tab 302 within each of the condition presets can be based on predetermined guidelines. Examples of predetermined guidelines can include: A Heart Failure preset condition that can include Weight, BP, Creatinine/eGFR, Potassium, Diuretic initiation/dosing, and RAS inhibitor. A Diabetes preset condition that can include BMI/weight, A1c, UACR, LDL-C, eGFR, and DM med dosing. An ASCVD preset condition that can include LDL-C, TG, hsCRP, Statin dosing, AST, and ALT. A HTN preset condition that can include SBP, DBP, Pulse, Creatinine, potassium, and BP meds. A CKD preset condition that can include BUN, Cr/eGFR, Urine protein, SBP, DBP, K/CO2, and ACE dosing. An Anemia (general) preset condition that can include Hgb, Ferritin, TSAT, B12, Folate, Iron supplements, B12 supplements, and Folate supplements. An Anemia in CKD preset condition that can include Hgb, Ferritin, TSAT, Iron dosing, and ESA dosing. A CKD bone and mineral preset condition that can include Ca, Phos, Vit D, PTH, Vit D dosing, AVD dosing, and Binder dosing. A Liver cirrhosis preset condition that can include Serum albumin, Bilirubin, Serum creatinine, INR, and Serum sodium. A HIV preset condition that can include CD4 count, Viral load, CBC, Creatinine, and HAART meds. A Hepatitis C preset condition that can include Hep C viral load, AST, ALT, and Serum albumin. A Lupus/lupus nephritis preset condition that can include Complement C3, Complement C4, dsDNA, WBC, plts, eGFR/creatinine, and Urine protein/creatinine ratio. A Thyroid preset condition that can include TSH, Free T4, and Synthroid dose. An Oncology/Chemotherapy preset condition that can include WBC, Hemoglobin, Platelets, Cr/eGFR, AST, ALT, and Serum albumin. The preset conditions can be defined to include any number of present conditions with any number of tests and are not intended to be limited and are provided for illustrative purposes only.

Referring to FIG. 3D, in response to receiving a selection of one of the conditions within the condition presets drop down menu 310, the GUI 300 can load characteristics for the selected condition and compare the characteristics against one or more files (e.g., a patient file) to see what results are included, are missing, or are missing but should be included. For example, in response to receiving a selection of the ASVID, the GUI 300 can load the tabs 302 and associated data lanes 306 for Total Cholesterol, Triglycerides, HDL Cholesterol, LDL Cholesterol, C-Reactive Proteins, AST (SGOT), and ALT (SGPT). Thereafter, data lanes 306 can each be populated with data points 320 from test results for a particular patient (i.e., Jane Doe).

Referring to FIG. 3E, in some embodiments, the GUI 300 can provide a missing observations notification 312 to show tests that are missing from a particular file or patient(s) for tests included within the selected condition presets drop down menu 310. For example, as shown in FIG. 3E, test results for Jane Doe's profile can be compared to tests associated with an ASCVD condition to determine that tests for LDL-P and Apo B are missing. The missing data can be conveyed to the user within the GUI 300 using any combination of methods. For example, as shown in FIG. 3E, a pop-up 330 can be generated and displayed to the user listing the missing tests.

Referring to FIG. 3F, in some embodiments, the GUI 300 can generate different types of charts, graphs, data, etc. depending on date types being visualized and/or user preferences. The different types of charts can include data point plotting, line graphs, bar graphs, graphic plotting, etc. and each can be plotted over a period of time (e.g., including a timeline 308). The different types of graphs being visualized by the GUI 300 can be adjusted based on information that is to be conveyed to the user and an optimal manner to convey that information. For example, as shown in FIG. 3F, a graph for Trigycerides results can be plotted as a plurality of data points 320 with a trend line 322, a graph for blood pressure can be plotted as a plurality of data point 320 pairs (e.g., systolic measurement and diastolic measurement) with a vertical line linking each pair and an ideal range line 324 for each data point 320 within the pair, a graph for body weight (BMI) can be plotted as a plurality of bar graph data points 320 with a trend line 322, and patient interactions can be plotted as a plurality of graphics representing types of interactions/information.

Referring to FIG. 3G, in some embodiments, the GUI 300 provides an interface that enables a user to interact with data points 320 within one or more data lanes 306 to view additional details about specific data entries. Each of the data points 320 populated within a data lane 306 can be linked to a data source for that data point 320 such that selection of the data point 320 can provide access to additional information related to the selected data point 320. In some embodiments, in response to receiving a selection of one of the data points 320 (e.g., via a mouse click), the GUI 300 can access a data source linked to the selected data point 320 and generate a pop-up showing details about the data point 320. For example, as shown in FIG. 3G, when a data point 320 within the Creatinine data lane 306 is selected, a pop-up 332 can be generated to show Creatinine test results for a point in time associated with the data point in greater detail. For example, the pop-up 332 can include a reference interval for Creatinine, a date of collection for the Creatinine test, a date the test result was reported, a status of the test, and the data source. The detailed pop-up 332 can include any combination of data that can be pulled from the data source for the data point 320. Each of the data points 320 can include their own set of detailed information.

Referring to FIG. 3H, in some embodiments, the GUI 300 provides an interface that enables a user to interact with and/or modify the visualization of data within the one or more data lanes 306. The visualization of data can be modified using any combination of operations. For example, as shown in FIG. 3H, in response to detecting a click and drag input, a highlighted window 334 can be generated within the GUI 300. In response to setting the highlighted window 334 (e.g., unclick a mouse button), the GUI 300 can emphasize the selected area of the data lane. The emphasis can include any combination of effects. For example, the emphasis can include zooming in on the selected area, pulling additional data for each of the data points within the selected area, highlighting or bolding data points within the selected area, etc. If a user would like to revert the GUI 300 back to a display prior to the highlighting, a reset button can be selected. The GUI 300 can provide any combination of interactions with the data lanes. For example, the GUI 300 can allow a user to scroll vertically or horizontally, zoom in or out, etc.

Referring to FIG. 3I, in some embodiments, a summary tab 316 can be accessed to provide additional contextual information to a user outside of a timelines view used by the data lanes 306. The summary tab 316 can be configured to expand in response to detecting an action from a user interacting with the GUI 300 (e.g., detecting a mouse click). Once expanded, in some embodiments, the summary tab 316 can reveal a plurality of collapsible headers. For example, the summary tab 316 can include collapsible headers for medications, encounters, procedures, and immunizations. The contextual information for the headers can include information about a particular patient relating to medication changes, encounters with an entity or other medical professionals, medical procedures undergone by the particular patient, immunizations administered to the particular patient, etc. Although these headers are provided, any combination of headers or categories can be included.

Referring to FIG. 3J, an example of one of the headers being expanded to show the additional contextual information is shown. The example in FIG. 3J is for a medications header and the contextual information shows medications taken during a given time period.

Referring to FIGS. 3A-3J, in some embodiments, the GUI 300 can include contextual data within one or more data lanes 306 along the timeline 308. For example, the patient medical history tabs 314 can include headers for contextual data related to interactions with and/or history of a patient. The patient medical history tabs 314 can be provided to convey any combination of patient/practitioner interactions. In some embodiments, the headers provided in the patient interaction tabs 314 can correspond to the collapsible headers provided in the summary tab 316. For example, the patient interaction tabs 314, and corresponding data lanes 306, can be provided to visualize patient observations, medications, procedures, encounters, immunizations, etc. over dates along the timeline 308. The medications can include medications prescribed to a patient or dates in which a medication has been taken by a patient. The procedures can include any medical procedures undertaken on the patient. The encounters can include interactions between the patient and a facility or physician. The immunizations can include any immunizations that the patient has received. In some embodiments, the contextual data associated with each of the patient medical history tabs 314 can be plotted against the timelines 308.

In some embodiments, each of the medications, procedures, encounters, immunizations, etc. can be plotted against the timelines 308 and each can include a unique combination of visual representations designating each event on a timeline 308. For example, medications can be represented by a pill graphic, immunizations by a syringe graphic, encounters by a calendar graphic, etc. In some embodiments, each of the graphics can be selected by a user to trigger additional information. For example, a selection of a pill graphic can be received and the GUI 300 can pull up information about the medication associated with the selected pill graphic for display to a user. In some embodiments, any of the graphics for the different contextual categories can include supplemental indicators. For example, if more than one medications, procedures, encounters, immunizations was undertaken on a given day, the corresponding graphic can be updated with a ‘+’ to indicate more than one occurrence for that date. Similar to the data points discussed above, user interaction with one of the graphics along the timeline 308 can cause the GUI 300 to generate a pop-up with additional information. For example, in response to a user selecting one of the pill graphics, the GUI 300 can trigger a pop-up showing any combination of status, medication taken, if/when the medication was taken, when the medication was prescribed, a dosage, medication instructions, side effects, etc.

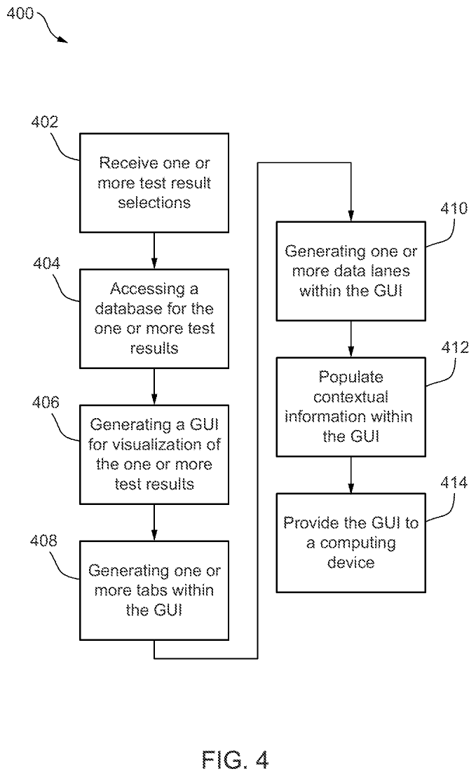

FIG. 4 provides an example flow chart depicting a process 400 in accordance with the aspects of the present disclosure. In particular, FIG. 4 depicts an illustrative process 400 for populating and interacting with a GUI including basic information, for example, GUIs 200 and 300, as depicted in FIGS. 2A-3J. The process 400 is discussed with respect to providing test results (or other types of results) for review by a user (e.g., a healthcare practitioner) and providing information related to those reports. However, the process 400 could be applied to any combination of industries and client/server relationships and is not intended to be limited to the examples provided herein. Initially, the GUI generates a list of test result categories 202 provided by an aggregation of test results from one or more data sources. The aggregation of test results can be performed in any combination of systems and methods. For example, the aggregation of test results can be provided as discussed in greater detail with respect to U.S. 63/387,859, which is incorporated by reference herein in its entirety.

Initially, a user is provided with a GUI depicting one or more options for tests to be visualized. The GUI can be provided by a service provider 106 through a web browser 110 or application on a client system 102. For example, the user can be provided with the GUI 200 depicting a list of tests, test panels, clinical groups, or a combination thereof. Alternatively, a user can load a visualizer 204 which may have a default starting state. For example, the user can be provided with the GUI 300 without any data populated within the tabs 302 or data lanes 306.

In some embodiments, the GUI 200, 300 receives an input for a target of the data to be visualized. For example, the GUI 200, 300 can receive a patient identification information (or other identifier) for a patient that the data is to be related to. The patient identification information (or other identifier) can be obtained through any combination of methods, for example, an input field. The patient identification information can include any combination of information about the patient. For example, the patient identification information can include the name of the patient, the date of birth of the patient, the medical record number of the patient, etc. The GUI 200, 300 can provide data that is specific to the patient to aid a medical professional in providing services to the patient. For example, GUI 300 can provide a trend capability that can visually plot data for that patient (e.g., chemistry or test data) and provide the data in a useful manner for aiding the medical professional in making one or more decisions.

At step 402, a selection of one or more tests is received. The one or more tests can be associated with a single patient and linked to the single patient based on an input received that includes patient identification information (or another identifier). The one or more test selections can be received through any combination of inputs. For example, the GUI 200 can present a list of selectable tests 202, test panels, and/or clinical groups 210. In some embodiments, the user can individually select one or more of the selectable tests as an input into the GUI 200. Input of the selected items can be executed in response to receiving activation of the visualizer button 204. Thereafter, the GUI 300 can be launched.

In some embodiments, the GUI 300 can be launched in a default initial state, without data populated, and the selection of one or more tests can be received in response to a selection of a condition preset and/or a clinical group(s) 210. In response to the selection of the condition preset and/or a clinical group(s) 210, the one or more tests associated therewith can be indexed. For example, in FIG. 2B, a selection has been input for a Comprehensive Metabolic Panel opening a subset of tests which include selections for Glucose and Creatinine. In this example, three selections can be provided as the selections, for example, upon selecting the visualizer 204 button. These selections can be made for a particular patient, for example, Jane Doe.

At step 404, a database containing the one or more test results is accessed to aggregate the test results associated with the selection(s) provided in step 402. The database can be, for example, consolidated database 120 from FIG. 1, record management system 104 from FIG. 1, or some combination thereof. The GUI 300 can use the selected tests to query the database for data tied to each of the selected tests. In some embodiments, accessing the database can include obtaining all data related to each of one or more tests.

Continuing the example from step 402, the database can be queried to access tables (or other data structure) for a Comprehensive Metabolic Panel associated with Jane Doe. The database can include a single database (consolidated database 120) or a collection of databases, including disparate data sources (e.g., client database 118, third party databases 122, service provider database 108) having information about Jane Doe. If the Comprehensive Metabolic Panel does not exist for Jane Doe, then an observations notification 312 can be updated with the selected Comprehensive Metabolic Panel and advance to a next query. Once the GUI 300 is loaded, the observations notification 312 can be provided, for example, within a selectable link for generating the observations notification 312 including any missing tests results for Jane Doe from the selected tests. If the query does return a result for the Comprehensive Metabolic Panel, then all of the data associated with Glucose and Creatinine can be aggregated and input into a visualization engine 114 for generation by a GUI. The database query can return all historical data associated with the selected test results, including tag(s) indicating a source database(s). For example, if Jane Doe has records for three Comprehensive Metabolic Panels within the accessed database(s), then all of the Glucose and Creatinine data in each of the Comprehensive Metabolic Panels can be aggregated to be visualized in the GUI 300. For each test result in the Glucose and Creatinine data, a data point can be generated.

In some embodiments, each data point can include a date of measurement and one or more measurement values, and, depending on the type of test result, biometric measurements, etc. associated with selected tests. For example, Jane Doe could have a Glucose test result taken on Feb. 4, 2013, with a value of 245, which will create a data point for that result at the appropriate date on the timeline. In some embodiments, each data point can be linked to the source of the information, such that when a data point is accessed, the detailed information can be provided, as discussed in greater detail herein. For example, a table can be generated or updated for each data point to include a linked data source.

At step 406, an initial GUI for visualizing the data points and other aggregated information is generated. Generating the initial GUI can involve, upon receiving the selection of one or more test results, automatically accessing a set of GUIs from a database (e.g. consolidated database 120 or record management system 104 from FIG. 1), selecting the initial GUI from the set of GUIs based on the selection, populating the initial GUI with data based on the selection, and rendering the initial GUI. The initial GUI can include basic structures to be populated with a combination of static data and/or dynamic data. For example, the initial GUI can be generated to include any combination of windows, menus, icons, controls, tabs, cursors, input fields, etc. In some embodiments, the initial GUI can be a basic structure for the GUI 300 and can include the basic structures for the condition preset menu 310, missing observations notification 312, tabs 302, 314, 316, data lanes 306, timeline 308, etc. Initially, certain elements can be generated with default values while others can be generated based on the data obtained from the database, as discussed in step 404.

At step 408, a category tab 302 for each of the selected one or more tests is generated. Generating the category tab 302 can involve, upon receiving the selection of the one or more tests, automatically accessing a set of category tab graphical elements from a database (e.g., consolidated database 120 or record management system 104 from FIG. 1), selecting a subset of the category tab graphical elements based on each test selection, and rendering the subset of category tab graphical elements. The number of category tabs 302 will be dictated on the number of selected tests, as long as there is sufficient data to be provided for each of the categories. For example, continuing the above example, if Glucose and Creatinine test results were selected, and Jane Doe's profile included data for both, a tab can be created for each test results category, as depicted in FIG. 3A. The generated category tabs 302 can include graphical elements, such as a header indicating a test result as well as supporting information for that category. For example, the Creatinine tab can include a Creatinine header and baseline information for that category. The baseline information can include, for example, the measurement values (e.g., mg/dL), measurement methods, ideal reference ranges (e.g., integer/multiple), etc. Each category can have a profile (e.g., in the consolidated database 120) with information to be displayed when that category is selected. The data for the category tabs 302 can be predetermined and may not be limited to data associated with a specific patient (e.g., EMR, patient profiles, test results, etc.).

At step 410, one or more data lanes 306, one for each of the category tabs 302 is generated. Generating each of the data lanes 306 can involve, upon generating each of the category tabs 302, automatically accessing data lane information from a database (e.g. consolidated database 120 or record management system 104 from FIG. 1), selecting a data lane 306 with parameters appropriate for the associated category tab 302, populating the data lane with data based on data in the associated category tab 302 or selection of tests, and rendering the data lane 306. In some embodiments, generating the data lanes 306 can include populating each data lanes 306 with data points 320, each of the data points can represent a test result received from the database. For example, the data lane 306 associated with the Creatinine tab, as shown in FIG. 3A, can include fourteen data points, with each data point representing a Creatinine test result for Jane Doe collected from the database(s). In some embodiments, each of the data points can be visualized to represent a disparate data sources from which data point originates. For example, a hallow outlined circle can represent a first database and a solid circle can represent a second database. Each data lane may include a different number of data points, depending on the number of test results for each category.

In some embodiments, any number of the data lanes 306 (i.e., 0-N) can include one or more trend lines 324 generated therein, reflecting an ideal reference range for the test results plotted in the data lane. As discussed herein, a style of the trend lines 324 can vary depending on the type of data being plotted, the type of measurement tool obtaining the test results, etc. Additionally, the trend lines 324 can be static or dynamic. For example, the trend line 324 for Glucose can be represented by a static horizontal line while the Creatinine trend lines 324 can be represented by variable separate vertical lines to represent different values obtained through different tools or methods. The data for the trend lines 324 can be obtained from predetermined values based on a category for the test results, measurement type, etc. and are not necessarily dictated by the test results themselves.

In some embodiments, instead of layering different data sets on top of one another, the data lanes 306 can each be plotted in substantially parallel data lanes 306 orientated substantially vertically to one another. Visualizing data in separate data lanes 306 vertically oriented to one another can enable a user to independently view and observe developments that would not otherwise be viewable. In some embodiments, patient medical history tabs 314 can be generated as substantially parallel data lanes 306 orientated substantially vertically to one another.

In some embodiments, the GUI can include a timeline 308. The dates with the timeline can be fixed or can be dynamic based on the data retrieved from the database. For example, if all the test results retrieved for Jane Doe fall within the time period of Feb. 4, 2013 and Aug. 8, 2022, then the timelines 308 will be generated with these starting and ending bookend dates with periodic dates listed between the two bookend dates. In some embodiments, the timeline 308 can act as an x axis for a generated graph visualization for a user. Each data point within each of the data lanes 306 can be identified, assigned an x value based on a date associated with the data point, assigned a y value based on a result, and automatically plotted on the graph visualization with the timeline 308 as the x axis. Data points from multiple data lanes 306 can be plotted on the same graph visualization. In some embodiments, the data points can be plotted according to data lanes 306, source databases, or based on user define parameters. For example, all data points associated with a first data lane can be plotted as squares and all data points associated with a second data lane can be plotted as circles. Additionally, each data point that originates from a first database can be plotted in blue, while each data point that originates from a second database can be plotted in red. The user can toggle over a data point to reveal additional information, such as specified x and y coordinates for the data point. Each of the data points within each of the data lanes can be populated according to the timeline 308 such that test results received at a same time (or close to the same time) will be viewable and vertically oriented to one another. Such a configuration can allow a user to easily view all results taken around the same period, for example, using a time indicator 328, to draw certain conclusions.

At step 412, contextual data for patient medical history tabs 314 can be populated based on information obtained from the database. The contextual data can be obtained and generated in a similar manner as the test result data for the category data lanes 306. In some embodiments, the data points 320 in the data lanes 306 for the patient medical history tabs 314 can have graphical representations associated therewith, for example, pills, charts, calendars, etc.

At step 414, a visualization of the data is displayed. For example, GUI 300 is displayed via a computing device (e.g., client system 102). Portions of the visualization of the GUI 300 can change depending on a type of data being displayed and data available (e.g., per patient/EMR). A source of the data can also vary depending on the type of data being displayed. For example, the data in the category tabs 302, the patient medical history tabs 314, the trend lines 324, and a condition preset can be based on predetermined data and can be consistent regardless of patient information being visualized in the GUI. Continuing this example, the data populated as data points within the data lanes 306, the values in the timeline 308, the missing observations in the missing observations notifications 312, and the contextual data in a summary tab 316 can be based on patient or EMR dependent data. Additionally, as discussed with respect to FIG. 3F, different types of data can be plotted in different data lanes 306.

As discussed herein, the various data visualized in the GUI 300 can also be collected and aggregated across one or more databases. Additionally, as a user interacts with the GUI 300, the visualization can be modified and/or updated. For example, a user can change what type of information is viewed, how the information is viewed, etc. by adjusting a tab modifier 304, selecting a different condition preset, highlighting a section 334 to be enhanced, selecting data points to display more information, expanding summary tab 316, adjusting the time indicator 328, etc.

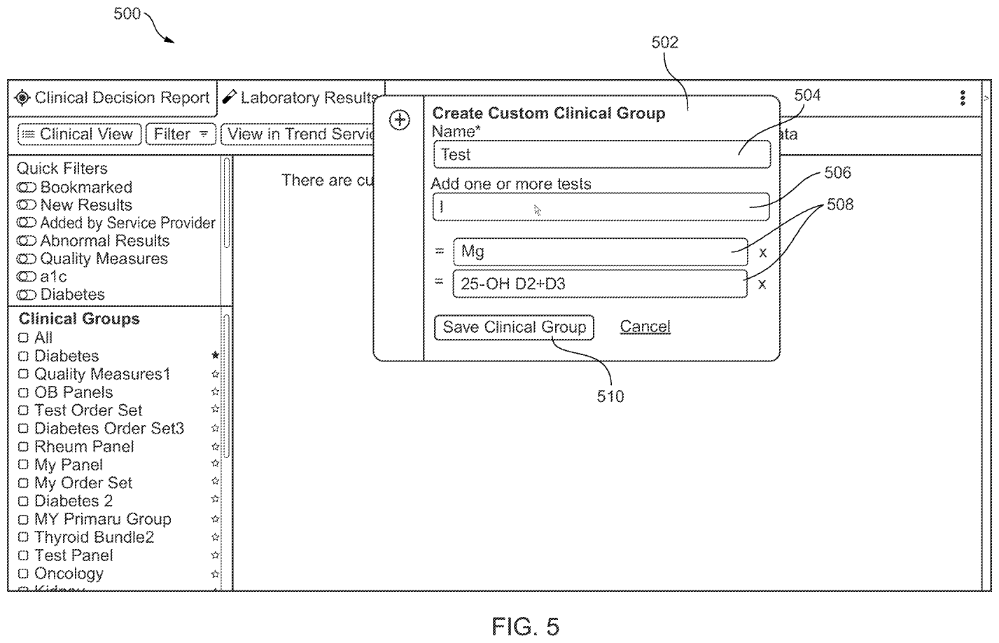

Referring to FIG. 5, in some embodiments, custom condition presets can be created for visualization by the GUI 300. For example, GUI 200 can provide a create custom group button that will generate a create custom group pop-up 502. In some embodiments, the create custom group pop-up 502 can include an input field 504 for inputting a name for a custom group, an input field 506 for inputting/searching for one or more categories, test types, chemistries, etc. to add to the custom group, one or more entries 508 that were added via the input field 506, and a save button 510 to save the custom group.

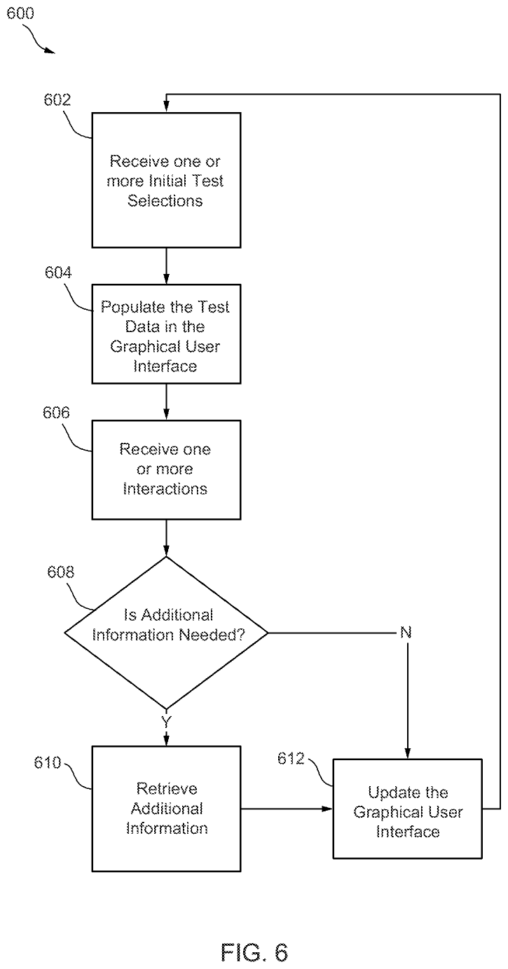

FIG. 6 provides an example flow chart depicting a process 600 in accordance with the aspects of the present disclosure. In particular, FIG. 6 depicts an illustrative process 600 showing operating steps for a user interacting with the GUIs 200, 300. The process 600 is discussed with respect to a healthcare practitioner requesting visualization of test results for a patient, however, the process 600 could be applied to any combination of industries and is not intended to be limited to the examples provided herein. The process 600 can be implemented using any combination of systems or methods discussed with respect to FIGS. 1A-5.

To initiate process 600 a user can access a visualization service providing a customizable and improved visualization of data. For example, a healthcare practitioner can access the GUIs 200, 300 provided by a service provider system 106 through a web browser 110 or other application to view one or more test results for one or more patients. The user can be provided with the GUI 200 or the GUI 300 and can access the different features of either GUI as discussed in greater detail with respect to FIGS. 1A-5.

At step 602, the process 600 involves receiving one or more initial test selections (or other data) to be visualized for the user. The one or more initial test selections can be associated with a single patient and linked to the single patient based on an input received that includes patient identification information (or another identifier). The selections can be received through any combination of methods, such as a selection of tests 202, test panels 208, condition preset 310, etc. The selections can be received via any combination of the selections within GUI 200 (as depicted in FIGS. 2A-2C) or within GUI 300 (as depicted in FIGS. 3A-3J). For example, receiving the selections of data to be visualized can include receiving a selection of one or more check boxes for tests 202 provided in GUI 200 or receiving a selection of a condition preset from a condition preset drop-down menu 310 provided in GUI 300. In some embodiments, the selection is provided to the system 106 in response to receiving an activation of a visualizer 204 button, for example, as provided in GUI 200, such that a user can make a number of selections or de-selections before providing the selections to the system 106 for processing (e.g., visualization).

At step 604, in response to receiving the one or more selections of tests, the process 600 involves updating the GUI 300 by visualizing the data associated with the selection(s). The GUI 300 can access a database containing one or more test results for each of the one or more tests and populate the GUI 300 using that data. The GUI 300 can be updated using any combination of methods, for example, using process 400 as discussed with respect to FIG. 4.

At step 606, the process 600 can involve receiving one or more interactions with the GUI 300. The interactions can include any combination of elements visualized within the GUI 300. For example, the interactions can include a user selection of a tab modifier 304, one or more of data lanes 306, a timeline 308, a condition presets drop down menu 310, a missing observations notification 312, patient medical history tabs 314, and a summary tab 316, one or more data points 320, trend line 322, ranges 324, time indicator 328, or a combination thereof. The selections can include other operations, for example, clicking and dragging, right clicking, left clicking, inputs from a keyboard or other input device, etc.

At step 608, the process 600 involves determining whether additional information is to be retrieved. Certain visualizations can involve extra information beyond information that is accessed, for example, at step 604. Based on the one or more interactions, the process can involve accessing additional information in a database (e.g. consolidated database 120 or record management system 104), selecting the additional information relevant to the one or more interactions, and rendering the information as part of a visualization. In some examples, such as when the interaction can be resolved with previously accessed data, no data may be retrieved. Depending on the selection(s) received, the process 600 can involve determining whether to retrieve additional information or to update the GUI 300 without any additional information. For example, if a data point 320 is selected, then additional information may be needed to generate the information provided in a pop-up 332 associated with the data point 320, whereas if a tab modifier 304 is selected, then the GUI 300 can be modified without retrieving additional information. The determination can be made using any combination of methods. The determination can be made based on a type of input received, the graphical element being selected, the type of data associated with a selected item, etc. If the selection dictates that additional information is needed, then the process 600 advances to step 610, if not the process advances to step 612.

At step 610, the process 600 involves initiating a query to a database, (e.g. consolidated database 120 or record management system 104), to request or retrieve additional information. The additional information can include any information needed to update the GUI 300. For example, if a data point 320 is selected, then a database associated with the data point 320 can be queried to collect information needed to generate a pop-up 332 for that data point 320.

At step 612, depending on the selection(s) received, the process 600 can involve updating the GUI 300. The GUI can show a visualization in a form of a graph depicting results of tests of possibly multiple categories from multiple database sources as a function of time. The data from multiple database sources can be aggregated and deduplicated so that each test result can have a single associated data point on the graph. The graph can include lines depicting values for normal test results and data on the graph can be distinguished by category and database source. The graph can allow the user to quickly assess testing trends for a specific patient and if needed, for each data point, the user can interact with the GUI 300. The GUI can generate a pop up window to show additional details about a selected data point to enrich the assessment. If advancing from step 608, the process 600 can involve modifying one or more visual elements of the existing GUI 300. For example, if the process 600 involves receiving a selection of a X on a tab modifier to close a tab 302, then that tab 302 and associated data lane 306 can be closed. If advancing from step 610, the process 600 can involve updating the GUI 300 using the retrieved information. For example, the GUI 300 will generate the pop-up 332 for the selected data point 320 and populate the pop-up 332 with the data retrieved from the appropriate database (e.g., as shown in FIG. 3G). The GUI 300 can be updated in any combination of ways depending on the selections and/or interactions received. For example, the GUI 300 can be updated by changing the data provided in the data lanes 106, expanding one or more areas of the GUI 300 to show additional information, modifying how the data is displayed (e.g., expanded data lanes), etc.

As noted herein, the flowcharts of FIGS. 4 and 6 illustrate the architecture, functionality, and operation of possible implementations of systems, methods, and computer program products according to various embodiments. In this regard, each block in the flowchart or block diagrams may represent a module, segment, or portion of code, which includes one or more executable instructions for implementing the specified logical functions. It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combination of blocks in the block diagrams and/or flowchart illustration, can be implemented by special purpose hardware-based systems that perform the specified functions or acts, or combinations of special purpose hardware and computer instructions.

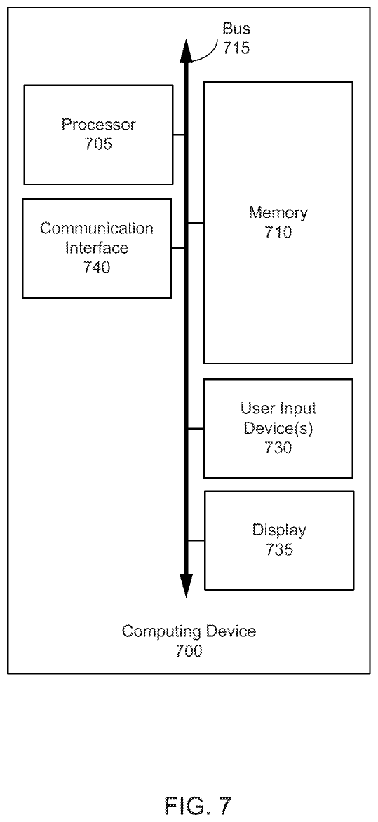

FIG. 7 illustrates an example computing device 700 suitable for use with systems and methods for generating credential enabled reference links for external resource access, including downloadable content, according to this disclosure. The example computing device 700 includes a processor 705 which is in communication with the memory 710 and other components of the computing device 700 using one or more communications buses 715. The processor 705 is configured to execute processor-executable instructions stored in the memory 710 to perform one or more methods according to different examples, such as part or all of the example processes 400, 600 described above with respect to FIGS. 4 and 6. In this example, the memory 710 stores processor-executable instructions that provide the various components and their related functionality, as discussed above with respect to FIGS. 1A-6. The computing device 700, in this example, also includes one or more user input devices 730, such as a keyboard, mouse, touchscreen, microphone, etc., to accept user input. The computing device 700 also includes a display 735 to provide visual output to a user such as a user interface.

The computing device 700 also includes a communications interface 740. In some examples, the communications interface 740 may enable communications using one or more networks, including a area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

While some examples of methods and systems herein are described in terms of software executing on various machines, the methods and systems may also be implemented as specifically-configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an algorithm-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example one or more non-transitory computer-readable media, that may store processor-executable instructions that, when executed by the processor, can cause the processor to perform methods according to this disclosure as carried out, or assisted, by a processor. Examples of non-transitory computer-readable medium may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

### ADDITIONAL CONSIDERATIONS

Although specific examples have been described, various modifications, alterations, alternative constructions, and equivalents are possible. Examples are not restricted to operation within certain specific data processing environments but are free to operate within a plurality of data processing environments. Additionally, although certain examples have been described using a particular series of transactions and steps, it should be apparent to those skilled in the art that this is not intended to be limiting. Although some flowcharts describe operations as a sequential process, many of the operations may be performed in parallel or concurrently. In addition, the order of the operations may be rearranged. A process may have additional steps not included in the figure. Various features and aspects of the above-described examples may be used individually or jointly.

Further, while certain examples have been described using a particular combination of hardware and software, it should be recognized that other combinations of hardware and software are also possible. Certain examples may be implemented only in hardware, or only in software, or using combinations thereof. The various processes described herein may be implemented on the same processor or different processors in any combination.

Where devices, systems, components or modules are described as being configured to perform certain operations or functions, such configuration may be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes may communicate using a variety of techniques including but not limited to conventional techniques for inter-process communications, and different pairs of processes may use different techniques, or the same pair of processes may use different techniques at different times.

Specific details are given in this disclosure to provide a thorough understanding of the examples. However, examples may be practiced without these specific details. For example, well-known circuits, processes, algorithms, structures, and techniques have been shown without unnecessary detail in order to avoid obscuring the examples. This description provides example examples only, and is not intended to limit the scope, applicability, or configuration of other examples. Rather, the preceding description of the examples will provide those skilled in the art with an enabling description for implementing various examples. Various changes may be made in the function and arrangement of elements.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific examples have been described, these are not intended to be limiting. Various modifications and equivalents are within the scope of the following claims.

In the foregoing specification, aspects of the disclosure are described with reference to specific examples thereof, but those skilled in the art will recognize that the disclosure is not limited thereto. Various features and aspects of the above-described disclosure may be used individually or jointly. Further, examples may be utilized in any number of environments and applications beyond those described herein without departing from the broader spirit and scope of the specification. The specification and drawings are, accordingly, to be regarded as illustrative rather than restrictive.

In the foregoing description, for the purposes of illustration, methods were described in a particular order. It should be appreciated that in alternate examples, the methods may be performed in a different order than that described. It should also be appreciated that the methods described above may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

Where components are described as being configured to perform certain operations, such configuration may be accomplished, for example, by designing electronic circuits or other hardware to perform the operation, by programming programmable electronic circuits (e.g., microprocessors, or other suitable electronic circuits) to perform the operation, or any combination thereof.

While illustrative examples of the application have been described in detail herein, it is to be understood that the inventive concepts may be otherwise variously embodied and employed, and that the appended claims are intended to be construed to include such variations, except as limited by the prior art.

What is claimed is:

1. A method for visualization of data, the method comprising:
receiving, by a processor, a selection of one or more tests;
accessing, by the processor, a database containing one or more test results for each of the one or more tests;
generating, by the processor, a category tab for each of the one or more tests; and
generating, by the processor, data lanes for each of the category tabs and populating each of the data lanes with one or more data points, each of the one or more data points representing the one or more test results received from the database.

2. The method of claim 1, wherein receiving the selection of the one or more tests comprises receiving a selection of a condition preset including one or more categories.

3. The method of claim 1, wherein receiving the selection of the one or more tests comprises receiving individual selections of each of one or more categories.

4. The method of claim 1, wherein the database comprises test results from a plurality of disparate data sources.

5. The method of claim 4, further comprising visualizing, by the processor, each of the one or more data points to indicate which of the plurality of disparate data sources that data point originates.

6. The method of claim 1, further comprising generating, by the processor, a missing observations notification including one or more test results missing from the database.

7. The method of claim 1, further comprising generating, by the processor, a graphical user interface (GUI) comprising a timeline, a condition presets drop down menu, and a missing observations notification.

8. A system comprising:
one or more processors; and
a memory coupled to the one or more processors, the memory storing a plurality of instructions executable by the one or more processors to perform operations comprising:
receiving a selection of one or more tests;
accessing a database containing one or more test results for each of the one or more tests;
generating a category tab for each of the one or more tests; and
generating data lanes for each of the category tabs and populating each of the data lanes with one or more data points, each of the one or more data points representing the one or more test results received from the database.

9. The system of claim 8, wherein the operation of receiving the selection of the one or more tests comprises receiving a selection of a condition preset including one or more categories.

10. The system of claim 8, wherein the operation of receiving the selection of the one or more tests comprises receiving individual selections of each of one or more categories.

11. The system of claim 8, wherein the database comprises test results from a plurality of disparate data sources.

12. The system of claim 11, the operations further comprising visualizing each of the one or more data points to indicate which of the plurality of disparate data sources that data point originates.

13. The system of claim 8, the operations further comprising generating a missing observations notification including one or more test results missing from the database.

14. The system of claim 8, the operations further comprising generating a graphical user interface (GUI) comprising a timeline, a condition presets drop down menu, and a missing observations notification.

15. A non-transitory computer-readable memory storing a plurality of instructions executable by one or more processors for causing the one or more processors to perform operations comprising:
receiving a selection of one or more tests;
accessing a database containing one or more test results for each of the one or more tests;
generating a category tab for each of the one or more tests; and
generating data lanes for each of the category tabs and populating each of the data lanes with one or more data points, each of the one or more data points representing the one or more test results received from the database.

16. The non-transitory computer-readable memory of claim 15, wherein the operation of receiving the selection of the one or more tests comprises receiving a selection of a condition preset including one or more categories.

17. The non-transitory computer-readable memory of claim 15, wherein the operation of receiving the selection of the one or more tests comprises receiving individual selections of each of one or more categories.

18. The non-transitory computer-readable memory of claim 15, wherein the database comprises test results from a plurality of disparate data sources.

19. The non-transitory computer-readable memory of claim 18, the operations further comprising visualizing each of the one or more data points to indicate which of the plurality of disparate data sources that data point originates.

20. The non-transitory computer-readable memory of claim 15, the operations further comprising generating a graphical user interface (GUI) comprising a timeline, a condition presets drop down menu, and a missing observations notification.
